(19) United States
(12) Patent Application Publication
(10) Pub. No.: US 2010/0036499 A1
(43) Pub. Date: Feb. 11, 2010

(54) KNEE PROSTHESIS

(75) Inventor: Vera Pinskerova, Prague (CZ)

Correspondence Address: Patent Docket Department;Armstrong Teasdale LLP, One Metropolitan Square, Suite 2600, St. Louis, MO, 63102-2740, US

(73) Assignee: Pinskerova

(21) Appl. No.: 12/296,832

(22) PCT Filed: Apr. 12, 2007
(86) PCT No.: PCT/IB2007/001618

§ 371 Date: Sep. 24, 2009

(30) Foreign Application Priority Data
Apr. 13, 2006 (GB) 0607544.4

(51) Int. Cl. A61F 2/38

(52) U.S. Cl. 623/20.31

(57) ABSTRACT

A knee prosthesis comprising a femoral component and a tibial component. The femoral component comprises a medial femoral condyle having a medial femoral condylar surface and a lateral femoral condyle having a lateral femoral condylar surface. The tibial component comprises a medial tibial condyle having a medial tibial condylar surface and a lateral tibial condyle having a lateral tibial condylar surface. The medial femoral condylar surface comprises a part-spherical convex surface and the medial tibial condylar surface comprises a part-spherical concave surface, the part-spherical surfaces being arranged to enable the medial femoral condyle to engage in sphere-in-sphere engagement with the medial tibial condyle. The sphere-in-sphere engagement provides anterior-posterior stability of the femoral component relative to the tibial component. The lateral tibial condylar surface comprises a track surface for the lateral femoral condyle to move across as the medial condyle pivots around the sphere-in-sphere engagement. The track surface is posteriorly unrestricted to permit the lateral femoral condylar surface to contact the track surface at a range of contact positions as the medial femoral condyle pivots relative to the medial tibial condyle around the sphere-in-sphere engagement.

### FIELD OF THE INVENTION

The present invention relates to a knee prosthesis, in particular a total knee prosthesis for a total knee replacement operation.

### BACKGROUND OF THE INVENTION

A natural knee connects the femur in the upper leg to the tibia in the lower leg. The natural knee joint can be considered as allowing two main types of movement: flexion-extension and tibial longitudinal rotation.

In flexion-extension movement the knee may be bent from a flexed, bent-legged, position to an extended, straight legged, position or vice verse. Full flexion is where the leg is bent to its maximum extent, which may be with the femur and tibia at an angle of 140 degrees, for example, although the actual angle will typically vary from person to person. Full extension is where the leg is straight, for example in a standing position.

Tibial longitudinal rotation is movement where the tibia rotates axially without also rotating the femur about its axis. In tibial longitudinal rotation there is some rotation of the tibia at the knee relative to the femur. This type of movement can be observed by bending one's knee, for example with the femur and tibia at a 90 degree angle, and moving one's toes from side to side through an arc.

A natural knee also provides anterior-posterior stability of the femur and tibia in relation to one another when the knee is in full extension.

Various attempts have been made to design knee prostheses which replicate the kinematics or functionality of the natural knee. A total knee prosthesis typically comprises two prosthetic components: a femoral component and a tibial component. In total knee replacement surgery, a surgeon typically surgically implants the prosthesis by replacing the ends of the femur and tibia with the femoral and tibial components respectively. Optionally, the patella (knee cap) may be replaced with a prosthetic patella component.

It is desirable to provide a knee prosthesis which, when implanted, provides kinematics or functionality which is, as far as possible, towards or equivalent to that of a natural knee joint.

GB 2 253 147 B describes a knee prosthesis which has an arcuate groove on the tibial component which permits, after implantation, limited anterior/posterior movement of the lateral side of the tibia relative to the femur in flexion of the knee joint.

The present invention seeks to provide an improved knee prosthesis.

### SUMMARY OF THE INVENTION

According to an aspect of the invention, there is provided a knee prosthesis comprising a femoral component comprising a medial femoral condyle having a medial femoral condylar surface and a lateral femoral condyle having a lateral femoral condylar surface; a tibial component comprising a medial tibial condyle having a medial tibial condylar surface and a lateral tibial condyle having a lateral tibial condylar surface; wherein the medial femoral condylar surface comprises a part-spherical convex surface and the medial tibial condylar surface comprises a part-spherical concave surface, the part-spherical surfaces being arranged to enable the medial femoral condyle to engage in sphere-in-sphere engagement with the medial tibial condyle; wherein the lateral tibial condylar surface comprises a track surface for the lateral femoral condyle to move across as the medial condyle pivots around the sphere-in-sphere engagement; and wherein the track surface is posteriorly unrestricted to permit the lateral femoral condylar surface to contact the track surface at a range of contact positions as the medial femoral condyle pivots relative to the medial tibial condyle around the sphere-in-sphere engagement.

By providing such an arrangement, the sphere-in-sphere engagement on the medial side provides relative anterior-posterior anchoring of the two components and allows pivotal flexion-extension movement. By providing the sphere-in-sphere engagement on the medial side in combination with a lateral tibial condylar surface comprising a track surface which is posteriorly unrestricted, kinematics or functionality which is towards or equivalent to that of a natural knee joint can be achieved. In particular, this arrangement allows the possibility of improved tibial longitudinal rotation movement and/or improved full flexion movement. Furthermore, embodiments of the invention can provide good anterior-posterior stability in full extension.

### BRIEF DESCRIPTION OF THE DRAWINGS

Embodiments of the present invention will now be described, by way of example only, with reference to the accompanying drawings in which like reference numerals are used to depict like parts. In the drawings:

FIG. 1 illustrates a frontal view of a knee prosthesis constructed in accordance with an embodiment of the invention;

FIG. 2a illustrates a side view of the knee prosthesis from the side indicated by line A-A in FIG. 1;

FIG. 2b illustrates a sagittal section of the knee prosthesis along line B-B of FIG. 1;

FIG. 2c illustrates a sagittal section of the knee prosthesis along line C-C of FIG. 1;

FIG. 3a illustrates the tibial component of the knee prosthesis in plan view;

FIG. 3b illustrates the relative movement allowed between the femoral and tibial components of the knee prosthesis;

FIGS. 4a to 4c are sagittal sectional views of the medial side of the knee prosthesis (as shown in FIG. 2b) illustrating the relative movement on the medial side allowed between the femoral and tibial components of the knee prosthesis;

FIGS. 5a to 5c are sagittal sectional views of the lateral side of the knee prosthesis (as shown in FIG. 2c) illustrating the relative movement on the lateral side allowed between the femoral and tibial components of the knee prosthesis, each of FIGS. 5a to 5c corresponding to the medial side equivalents shown in FIGS. 4a to 4c respectively;

FIGS. 5d and 5e are further sagittal sectional views of the lateral side of the knee prosthesis illustrating the possible relative movement on the lateral side allowed between the femoral and tibial components of the knee prosthesis as the knee bends further;

FIGS. 6a and 6b are further sagittal sectional views of the lateral side of the knee prosthesis illustrating other possible relative movement on the lateral side allowed between the femoral and tibial components of the knee prosthesis;

FIG. 7a is a medial-lateral sectional view illustrating the medial-lateral profile of the track and lateral femoral condylar surface;

FIGS. 7b and 7c are medial-lateral sectional views illustrating alternative medial-lateral profiles of the track and lateral femoral condylar surface;

FIG. 8a is a plan view of the tibial component illustrating the medial-lateral track profile and lateral side profile of the intercondylar eminence;

FIGS. 8b to 8d are plan views of the tibial component illustrating alternative medial-lateral track profiles and alternative lateral side profiles of the intercondylar eminence;

FIGS. 9a and 9b are sagittal sectional views of the lateral side, illustrating an alternative structure for the lateral side of the femoral component;

FIG. 10 is a sagittal sectional view of a tibial component with a posterior surface which extends distally beyond the base; and

FIGS. 11a to 11i are sagittal sectional views of tibial components, corresponding to the sagittal sectional views of FIGS. 2c, 5a to 5e and 6a to 6b and illustrating alternative anterior-posterior profiles of the track.

### DETAILED DESCRIPTION

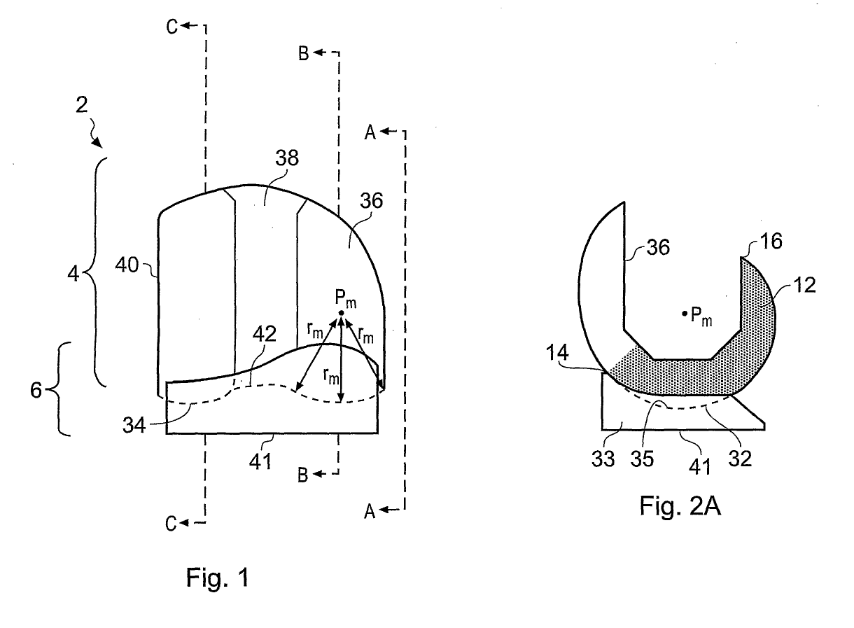

The embodiments of FIGS. 1 to 11 and the following description relate to a total knee prosthesis for a right knee. The corresponding prosthesis for a left knee (not shown) is a mirror image. Also, where particular dimensions are given in relation to these figures, these are typically for a European of height around 1.8 metres.

In all of the sagittal sectional views, as shown, the anterior side is on the left and the posterior side is on the right. “Medial” refers to the inner part of the knee prosthesis (when in the implanted orientation) and “lateral” refers to the outer part.

The figures which illustrate movement of the knee prosthesis, illustrate the potential movement allowed by the prosthesis when implanted.

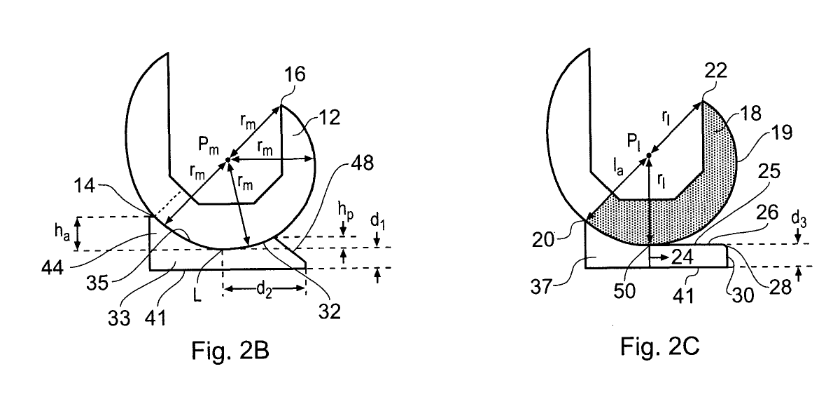

Referring to FIGS. 1 and 2a to 2c, FIG. 1 is a frontal view of a knee prosthesis 2, FIG. 2a illustrates a side view from the side indicated by line A-A in FIG. 1, FIG. 2b illustrates a sagittal section along line B-B of FIG. 1; and FIG. 2c illustrates a sagittal section along line C-C of FIG. 1.

The knee prosthesis 2 comprises a femoral component 4 which in turn comprises a medial femoral condyle 12 (shown in FIGS. 2a and 2b and shaded in FIG. 2a) having a medial femoral condylar surface 32 (also shown in FIGS. 2a and 2b). The femoral component 4 also comprises a lateral femoral condyle 18 (shown and shaded in FIG. 2c) which has a lateral femoral condylar surface 19 which extends from point 20 to point 22.

The prosthesis 2 also comprises a tibial component 6 comprising, with reference to FIGS. 2a and 2b, a medial tibial condyle 33 having a medial tibial condylar surface 35. The tibial component also comprises, with reference to FIG. 2c, a lateral tibial condyle 37 having a lateral tibial condylar surface 25.

The medial femoral condylar surface 32 comprises a part-spherical convex surface 32. With reference to FIG. 2b, this surface 32 is part-spherical in that the surface profile is part-circular in sagittal section and part-circular in lateral-medial section. That is, in the sagittal section of FIG. 2b the surface is part-circular from point 14 to posterior point 16 around a centre point Pₘ with a radius rₘ in a range from 20 to 25 mm. In lateral-medial section, and with reference to FIG. 1 which shows as dashed line 34 the contact at its lowermost point between the two components, the surface 32 is part-circular around point Pₘ as shown.

The medial tibial condylar surface 35 comprises a part-spherical concave surface 35. The part-spherical surfaces 32, 35 are arranged to enable the medial femoral condyle 12 to engage in sphere-in-sphere engagement with the medial tibial condyle 33. The sphere-in-sphere engagement permits pivoting movement around a substantially fixed point Pₘ to allow flexion-extension movement and tibial longitudinal rotational movement. The sphere-in-sphere engagement also provides relative anterior-posterior fixing of the two components and provides anterior-posterior stability. The part-spherical outer concave surface is typically a clearance fit for the part-spherical inner convex surface.

In the depicted embodiment, the tibial component 6 has an intercondylar eminence 42 which projects between the tibial lateral condylar surface 25 and the tibial medial condylar surface 35.

In the depicted embodiment the femoral component also comprises an anterior flange formed by shoulders 36 and 40 and midline groove 38. Anterior flanges of known configuration may be used. The midline groove 38 is arranged to receive the patella and may be angled upwards and laterally at around a 5 degree angle. Viewed from the side in sagittal section the groove has a floor with a single radius. The groove and intercondylar eminence 42 are configured so that the floor of the groove moves freely over the intercondylar eminence as the femoral and tibial components move relative to each other.

As shown in FIG. 1, the lateral shoulder 40 typically extends higher than the medial shoulder 36 and each shoulder blends into the profile of the respective femoral lateral or medial condylar surface.

The tibial component has a base 41 which defines a plane. A point L (see FIG. 2b) on the concave surface nearest to the plane of the base is a distance d₂ in a range from 20 to 25 mm from the posterior surface of the medial side of the tibial component. By matching this distance d₂ to the radius rₘ of the part-spherical convex surface 32 of the medial femoral condylar surface 32, the medial side of the femoral and tibial components may be posteriorly aligned.

With reference to FIGS. 2b and 2c, a posterior part of the lateral femoral condylar surface (from point 50 to point 22 in FIG. 2c) is circular in sagittal section with a radius r₁. The part-spherical convex surface 32 of the femoral medial condyle 12 (FIG. 2b) has a radius rₘ. In the depicted embodiment r₁ is about 2 mm smaller than rₘ.

FIG. 2b also shows a thickness of the medial side of the tibial component as the distance d₁ between point L and the base 41. In the depicted embodiment this distance is about 6 mm, but can vary as required in other embodiments.

Referring to FIG. 2c, the lateral tibial condylar surface 25 comprises a track surface 26 for the lateral femoral condyle 18 to move across as the medial condyle 12 pivots around the sphere-in-sphere engagement. The track surface 26 is posteriorly unrestricted to permit the lateral femoral condylar surface 19 to contact the track surface 26 at a range of contact positions as the medial condyle 12 pivots around the sphere-in-sphere engagement.

The track surface can be considered as extending from a position defined by the contact position 50 between the lateral femoral condylar surface 19 and the lateral tibial condylar surface 25 at full extension, as depicted in FIG. 2c, to a position defined by the posterior extreme (the rightmost extreme in FIG. 2c) of the lateral tibial condylar surface 25. Arrow 24 in FIG. 2c illustrates the anterior beginning of track surface 26.

In the embodiment of FIG. 2c, the track surface 26 comprises a planar portion 26 which is planar along the track (posteriorly from point 50) in the anterior-posterior direction. The base 41 of the tibial component defines a plane and in the depicted embodiment the planar portion is parallel to the plane.

FIG. 2c also shows a thickness of the lateral side of the tibial component as the distance d₃ between the tibial condylar surface 25 and the base 41. In the depicted embodiment this distance is about 6 mm, but can vary as required in other embodiments.

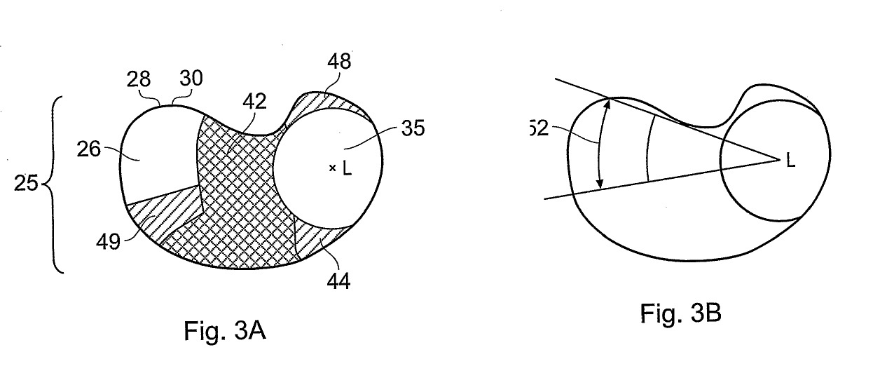

Referring now to FIG. 3a, this figure illustrates the tibial component of the knee prosthesis in plan view. The part-spherical concave surface 35 in which the femoral medial condyle 12 engages is shown. The surface 35 curves down to a lowermost point L in the centre as shown. The surface 35 is defined at least in part by an anterior lip 44 and a posterior lip 48 (the surface 35 is also defined by part of the intercondylar eminence 42). The lips 44 and 48 and intercondylar eminence 42 blend into the part-spherical surface.

As can be seen from FIGS. 3a and 2c, the lateral tibial condylar surface 25 has a concave anterior restriction 49 arranged to contact a corresponding restriction part (the part between points 20 and 50 in FIG. 2c) of the lateral femoral condylar surface 19 in full extension.

The restriction part (from point 20 to point 50) of the lateral condylar surface is anterior to a posterior part (from point 50 to point 22 in FIG. 2c) of the lateral femoral condylar surface. As shown in FIG. 2c, the posterior part is circular in sagittal section from its posterior end 22 to a position 50 which contacts the lateral tibial condylar surface 25 in full extension. The restriction part of the lateral femoral condylar surface has a larger radius, depicted as lₐ, in sagittal section than the radius r₁ of the posterior part. In full extension this configuration, in combination with the lateral co-lateral ligament and posterior capsule restricts the implanted knee prosthesis from excessive tibial external rotation and hyperextension. The latter function is understood to be provided mainly by the medial side of the natural knee, but the sphere-in-sphere engagement of the medial side of the depicted knee prosthesis does not mimic this part of the natural knee.

FIG. 3b illustrates the relative movement allowed between the femoral and tibial components of the knee prosthesis. The medial femoral condyle and medial tibial condyle are arranged to pivot relatively to one another around a central point of the sphere-in-sphere engagement as the lateral femoral condyle moves across the track 26. The movement is depicted by arrow 52 in FIG. 3b.

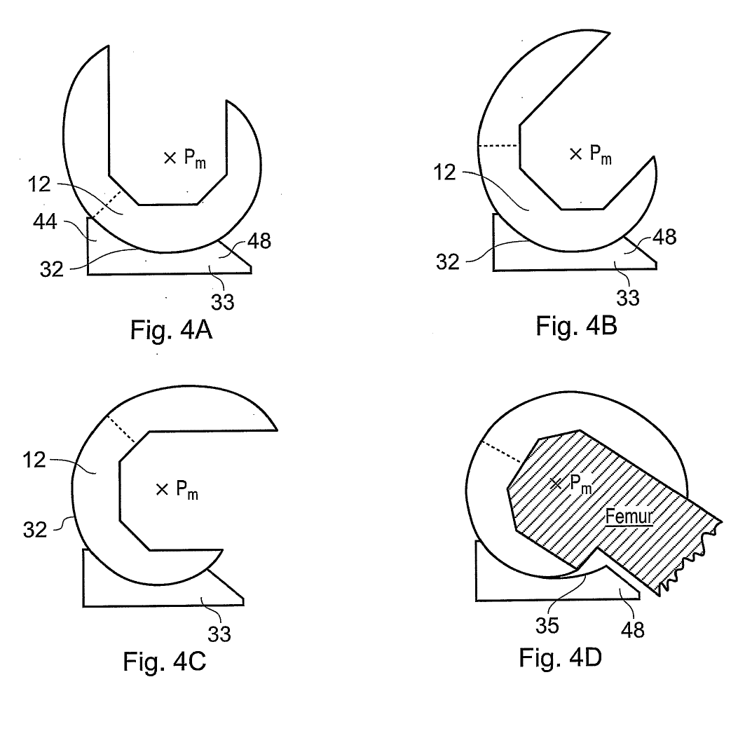

FIGS. 4a to 4d are sagittal sectional views of the medial side of the knee prosthesis (as shown in FIG. 2b) illustrating the relative movement on the medial side allowed between the femoral and tibial components of the knee prosthesis as the knee moves through a flexion-extension movement. FIG. 4a shows the knee prosthesis in full extension, FIG. 4b shows the knee prosthesis with the leg bent slightly (at around 45 degrees), FIG. 4c shows the knee bent further (at around 90 degrees) and FIG. 4d shows the knee prosthesis bent further still (at around 140 degrees). It can be seen from these figures how the medial femoral condyle 12 pivots around point Pₘ relative to the medial tibial condyle 33.

FIG. 4d shows the femur (shaded in the figure) and how the lower posterior lip 48, which is posteriorly inclined downwards in a region which is posterior to the concave surface 35, is arranged to permit around 140 degree flexion movement. This figure also illustrates how the femur itself has been transected.

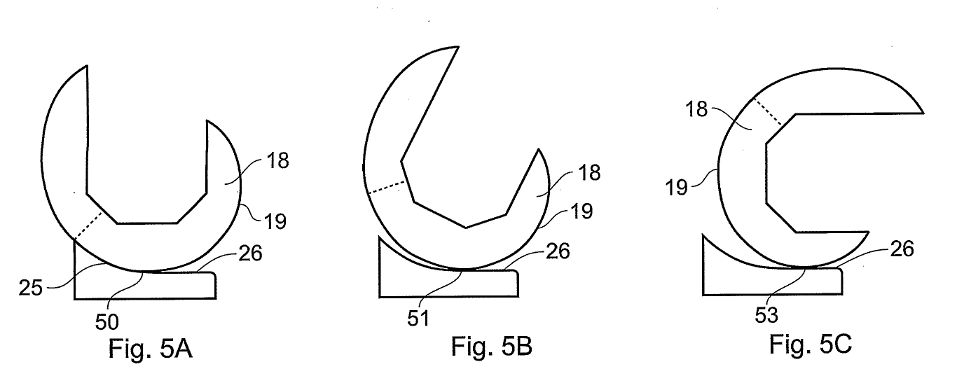

FIGS. 5a to 5c are sagittal sectional views of the lateral side of the knee prosthesis (as shown in FIG. 2c) illustrating the relative movement on the lateral side allowed between the femoral and tibial components of the knee prosthesis. Each of FIGS. 5a to 5c correspond to the medial side equivalents shown in FIGS. 4a to 4c respectively. Accordingly, FIG. 5a shows the knee prosthesis in full extension, FIG. 5b shows the knee prosthesis with the leg bent slightly (at around 45 degrees) and FIG. 5c shows the knee bent further (at around 90 degrees). It can be seen how the lateral femoral condyle 18 pivots and moves posteriorly along track 26 at a range of contact positions 50, 51 and 53. The track surface 26 is posteriorly unrestricted, allowing the lateral femoral condyle 18 to move posteriorly as far as full tibial internal rotation and flexion require. This arrangement allows the possibility of improved tibial longitudinal rotation movement and/or improved full flexion movement.

FIGS. 5d and 5e are further sagittal sectional views of the lateral side of the knee prosthesis illustrating the possible relative movement on the lateral side allowed between the femoral and tibial components of the knee prosthesis as the knee bends further to around 140 degrees, corresponding to a medial side view such as that shown in FIG. 4d. Referring to these figures, the track surface 26 has a posterior extreme at the posterior extreme of the lateral tibial condylar surface and the tibial component comprises a rounded portion 28 which connects the posterior extreme of the track surface to the posterior surface 30 of the lateral side of the tibial component. The range of contact positions which the lateral femoral condylar surface can contact the track surface include (i) a contact position 55 at the posterior extreme of the track surface, and/or (ii) a contact position 57 on the rounded portion 28.

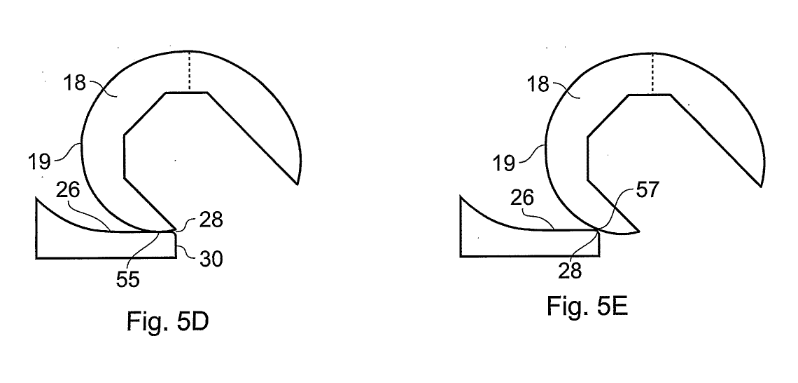

The knee prosthesis may allow other relative movement between the femoral and tibial components, for example the movement shown in FIGS. 6a and 6b which are further sagittal sectional views of the lateral side of the knee prosthesis. As can be seen from these figures, the lateral femoral condyle 18 may lose contact with the track surface 26 (while the medial side sphere-in-sphere engagement remains engaged). FIG. 6b shows how the prosthesis may allow full flexion movement in some patients. The actual relative movement for a particular patient will depend on a range of considerations, not least the inherent anatomical constraints of the individual. In other words the prosthesis may permit certain movements, but a particular patient may not make use of the full extremes of the movements permitted.

Embodiments may allow around 140 degree flexion movement and 30 degree longitudinal rotational movement.

As the lateral side of the knee prosthesis permits posteriorly unrestricted movement, the medial side has been arranged to accommodate this movement. Referring back to FIGS. 3a and 2b, the tibial component has a base which defines a plane and the medial tibial condylar surface has an anterior lip 44 and a posterior lip 48 which at least partly define the part-spherical concave surface. The anterior lip is higher than the posterior lip, when the height of each lip is measured from the plane of the base. The lower posterior lip 48 permits improved flexion movement whilst the higher anterior lip 44 provides posterior tibial stability.

Referring to FIG. 2b, when the height of each lip is measured from a plane which is parallel to the plane of the base and which contains the point L on the concave surface nearest to the plane of the base the height of the anterior lip hₐ is about 10 mm and the height of the posterior lip h₁ is about 3 mm in the depicted embodiment.

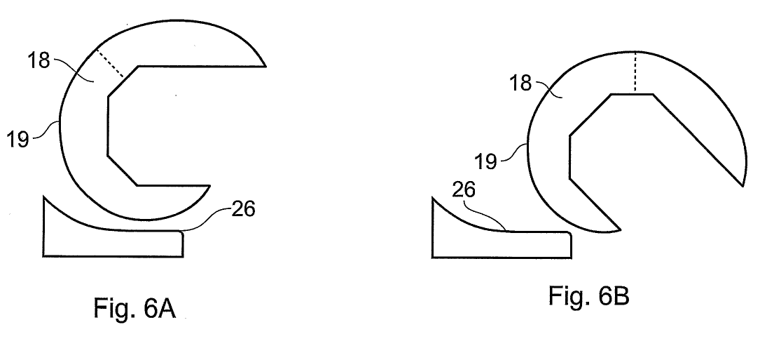

FIG. 7a is a medial-lateral sectional view illustrating the medial-lateral profile of the track 26 and lateral femoral condylar surface 19. As can be seen the medial-lateral profile of the track surface 26 conforms with the medial-lateral profile of the lateral femoral condylar surface 19 and the intercondylar eminence 42. Some clearance (not shown) may be provided between the lateral femoral condyle and the intercondylar eminence 42 to permit the pivoting movement around the medial side. The lateral tibial side of the prosthesis may end at any one of points 70, 72 or 74. That is, the lateral tibial side of the prosthesis may extend laterally beyond the lateral extreme of the femoral component.

In embodiments where the lateral tibial side of the prosthesis extends laterally beyond the lateral extreme of the femoral component the tibial surface 76 on the lateral side of the track surface 26 may be flat. This can make this part of the prosthesis simple to manufacture.

FIGS. 7b and 7c are medial-lateral sectional views illustrating alternative medial-lateral profiles of the track surface 26 and lateral femoral condylar surface 19. Again, the medial-lateral profile of the track surface 26 conforms with the medial-lateral profile of the lateral femoral condylar surface 19 and the intercondylar eminence 42; and the lateral tibial side of the prosthesis may end at any one of points 70, 72 or 74.

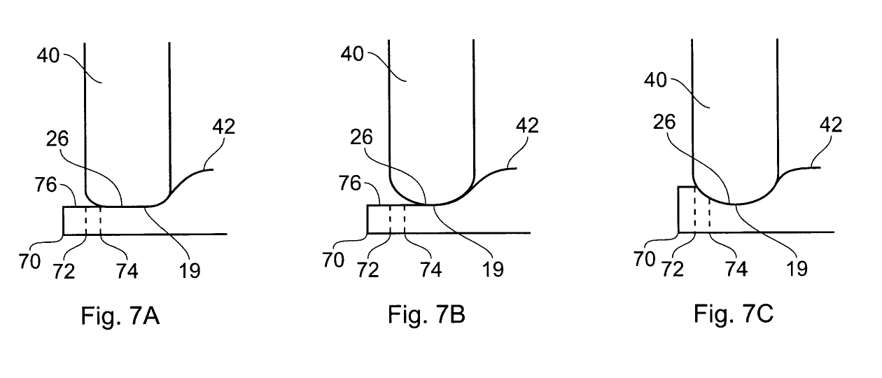

Using the definitions of Yoshioka Y, Siu D W, Scudamore R A et al, “Tibial Anatomy and Functional Axes”, Journal of Orthopaedic Research, Vol. 7, defined. A medial-lateral axis aₘ₋ₗ and anterior-posterior axis aₐ₋ₚ are depicted in FIG. 8a. FIG. 8a is a plan view of the tibial component illustrating the medial-lateral track profile and lateral side profile 80 of the intercondylar eminence 42. Here, the posterior surface of the lateral side of the tibial component is nearer to the medial-lateral axis aₘ₋ₗ than the posterior surface of the medial side of the tibial component. In the illustrated embodiment, the posterior surface of the lateral side of the tibial component is about 7 mm nearer to the medial-lateral axis aₘ₋ₗ than the posterior surface of the medial side of the tibial component. That is, in FIG. 8a d₁ is about 7 mm less than dₘ.

In FIG. 8a, the track surface 26 extends from a position defined by the contact position between the lateral femoral condylar surface and the lateral tibial condylar surface at full extension to a position defined by the posterior extreme of the lateral tibial condylar surface. In the depicted embodiment, the anterior-posterior length lₜ of the track is about 25 mm.

The intercondylar eminence 42 between the lateral tibial condylar surface and the medial tibial condylar surface has a lateral side 80 where the intercondylar eminence 42 joins the track surface 26. The lateral side 80 profiled to allow the pivotable movement of the lateral femoral condyle across the track surface, and in FIG. 8a is a part-circumferential profile.

In FIG. 8a concave anterior restriction 49 is shown (which is not present in the alternatives shown on FIGS. 8b to 8d).

FIGS. 8b to 8d are plan views of the tibial component illustrating alternative medial-lateral track profiles and alternative lateral side profiles of the intercondylar eminence. In FIG. 8b, the tibial lateral condylar surface 25 is planar across its entire anterior-posterior extent and the lateral side 80 has a part-circumferential profile.

FIGS. 8c and 8d show the lateral side (84, 86, 88) of the intercondylar eminence 42 having a profile which is a combination of at least one tangential profile 84, 88 and at least one part-circumferential profile 86.

The tangential profiles 84, 88 can provide an element of controlled knee movement and can be selected to achieve a particular pivotal movement profile with improved stability.

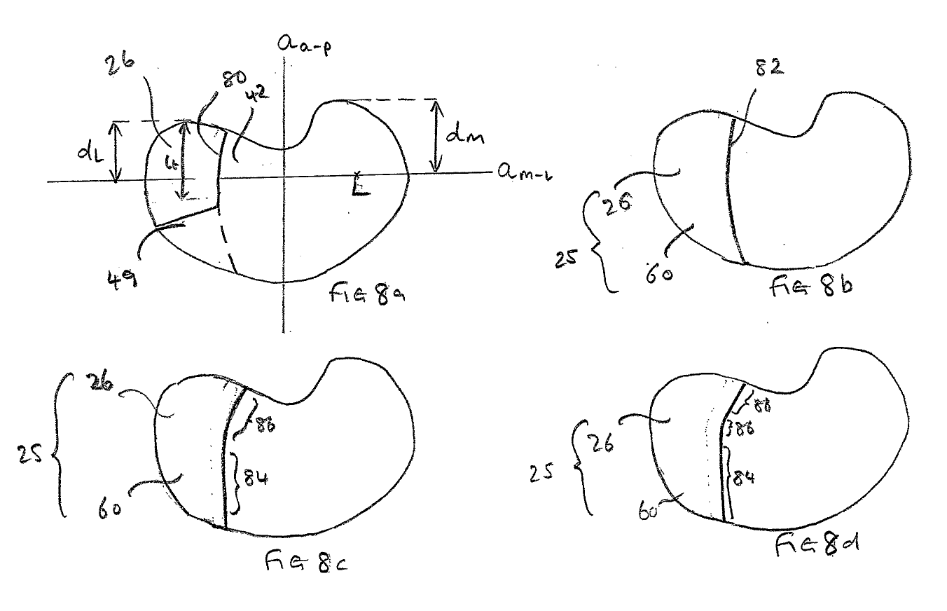

FIGS. 9a and 9b are sagittal sectional views of the lateral side, illustrating an alternative structure for the lateral side of the femoral component.

In FIGS. 9a and 9b, the lateral tibial condylar surface 25 has planar anterior surface 60 and the lateral femoral condylar surface 19 has a planar formation 92 which is arranged to contact the planar anterior surface 60 of the lateral tibial condylar surface 25 in full extension (depicted in FIG. 9b).

As can be seen, the planar formation 92 on the lateral femoral condylar surface is anterior to a posterior part of the lateral femoral condylar surface which is circular in sagittal section from its posterior end 22 to a position 50 which contacts the lateral tibial condylar surface in full extension. Also, as can be seen from FIGS. 9a and 9b the lateral tibial condylar surface 25 is planar from its anterior extreme to its posterior extreme (and comprises planar portion 92 and track surface 26).

In FIGS. 9a and 9b, the additional part 90 of the femoral component when compared with the embodiment of FIG. 2c is shaded.

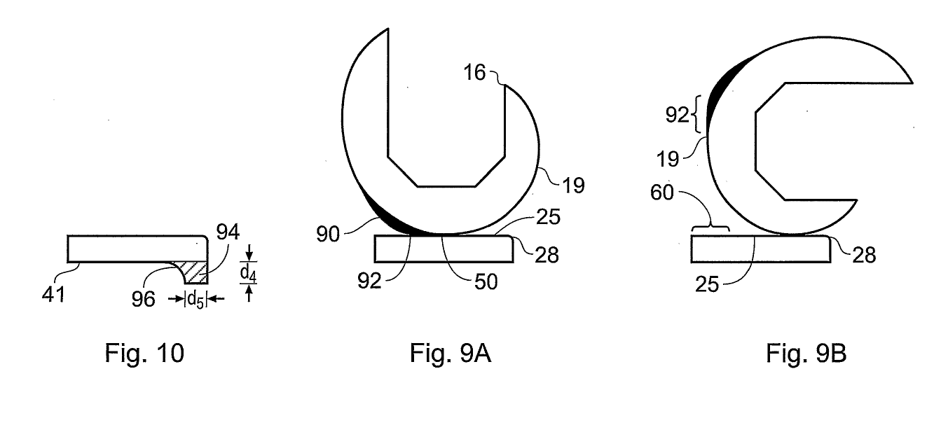

FIG. 10 is a sagittal sectional view of a tibial component with a posterior surface which extends distally beyond the base 41. The posterior surface of the lateral side of the tibial component extends distally beyond the plane of the base as shown by extension 94. Distances d4 and d5 are about 6 mm and the join 96 between base 41 and extension 94 is curved to account for possible stress. The extension is provided to allow the lateral femoral condyle 18 (c.f. FIGS. 5e and 6b) to move around the posterior edge of the prosthesis without directly contacting, for example, part of a backing plate used to fix the tibial component to the tibia.

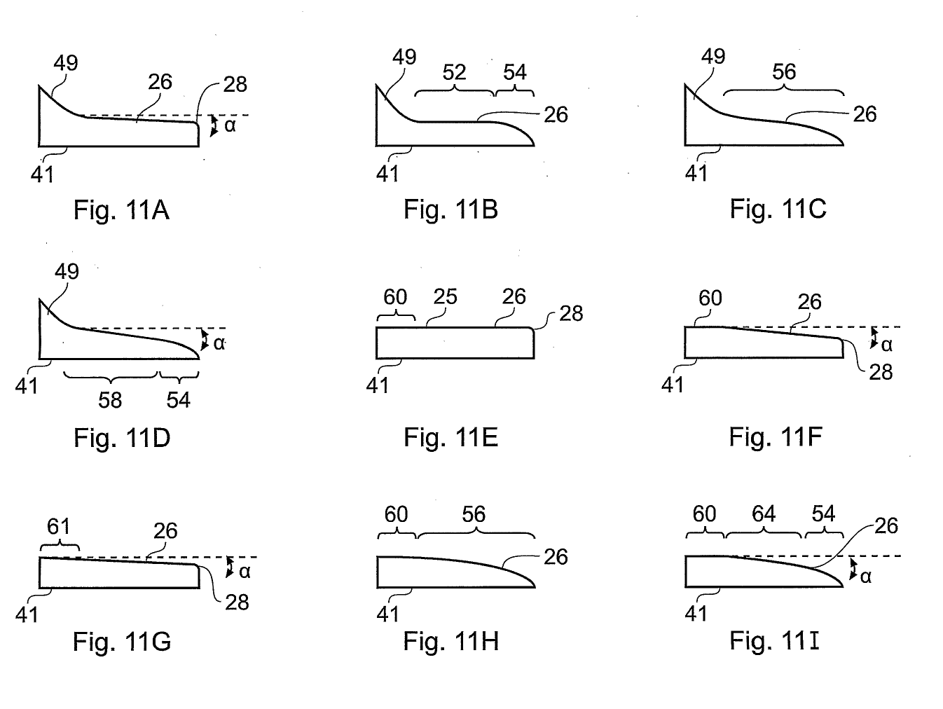

FIGS. 11a to 11i are sagittal sectional views of the lateral side of tibial components, corresponding to the sagittal sectional views of FIGS. 2c, 5a to 5e and 6a to 6b and illustrating alternative anterior-posterior profiles of the track in embodiments which are variations of the knee prosthesis of FIG. 1.

FIG. 11a illustrates a tibial component with a concave anterior restriction 49 (as discussed in relation to FIG. 3a). The component with a base 41 which defines a plane and a track 26 including a planar portion which is inclined posteriorly downwards at angle alpha to the plane.

FIG. 11b illustrates a tibial component with a concave anterior restriction 49, a planar portion 52 parallel to base 41 and a posterior portion 54 which is convex.

FIG. 11c illustrates a tibial component with a concave anterior restriction 49, and a track 26 formed from a posterior portion 56 which is convex.

FIG. 11d illustrates a tibial component with a concave anterior restriction 49, a posteriorly inclined planar portion 58 and a posterior portion 54 which is convex.

FIG. 11e illustrates a tibial component which is planar in the anterior-posterior direction across its entire length i.e. across the whole tibial lateral condylar surface 25, including a planar anterior surface 60.

FIG. 11f illustrates a tibial component with a planar anterior surface 60 and a posteriorly downwardly inclined planar track surface 26.

FIG. 11g illustrates a tibial component with an inclined surface across the entire anterior-posterior length, including an inclined planar anterior surface 61 and a posteriorly downwardly inclined planar track surface 26.

FIG. 11h illustrates a tibial component with a planar anterior surface 60 and a posterior portion 56 which is convex.

FIG. 11i illustrates a tibial component with a planar anterior surface 60, an inclined planar portion 64 and a posterior portion 54 which is convex.

The posterior extreme of the lateral tibial surface is rounded as rounded portion 28 in FIGS. 11a, 11e, 11f and 11g.

In FIGS. 11a, 11d, 11f, 11g and 11i, the tibial component has a base 41 which defines a plane and the planar portion is inclined posteriorly downwards, at an angle alpha, relative to the plane. In various embodiments the planar portion 26, 58, 64 is inclined up to 12 degrees posteriorly downwards relative to the plane of the base, optionally in a range from 8 degrees to 12 degrees and further optionally about 10 degrees.

It will be appreciated that in various embodiments the profiles of FIGS. 11a to 11i can be appropriately combined with those of FIGS. 7a to 7c, FIGS. 8a to 8d and FIGS. 9a and 9b.

As is known in the art the knee prosthesis can be fabricated from mechanically and physiologically suitable materials including ceramics, metals and polymers. In one embodiment the femoral component is manufactured from a suitable metal or alloy and the tibial component is manufactured from ultra high molecular weight polyethelene.

As will be appreciated the knee prosthesis may be implanted by affixing, with or without “cement” (polymethyl methacrylate), a femoral component to a femur and affixing, with or without cement, a tibial component to a tibia. There are various methods for fixing the components in place including metal or plastic pegs, which may be integral with the femoral component or integral with a backing plate for the tibial component.

Embodiments of the invention have been described by way of example only. It will be appreciated that variations of the described embodiments may be made which are still within the scope of the invention.

For example, to accommodate larger or smaller individuals, the anterior lip of the medial tibial condylar surface may have a height of 7 mm to 13 mm and the posterior lip may have a height in a range from 1 mm to 5 mm. Optionally, the height of the anterior lip may be in a range from 9 mm to 11 mm and the height of the posterior lip may be in a range from 2 mm to 4 mm. Similarly, the radius r₁ may be in a range of 1 mm to 3 mm smaller than rₘ and/or the posterior surface of the lateral side of the tibial component may be in a range from 5 mm to 9 mm nearer to the medial-lateral axis than the posterior surface of the medial side of the tibial component. Similarly, anterior-posterior length (lₜ) of the track may be in a range from 20 mm to 30 mm and optionally in a range from 22 mm to 28 mm.

Also, for example, the tibial component may be of larger or smaller area achieved by adding material medially, laterally or anteriorly to provide full coverage of the transected tibia.

Also, for example, the thickness of the tibial component may vary to accommodate the level of tibial cut.

Also, with regard to the femoral component the anterior-posterior length may vary to fit the femur.

1. A knee prosthesis comprising:
a femoral component comprising a medial femoral condyle having a medial femoral condylar surface and a lateral femoral condyle having a lateral femoral condylar surface;
a tibial component comprising a medial tibial condyle having a medial tibial condylar surface and a lateral tibial condyle having a lateral tibial condylar surface;
wherein the medial femoral condylar surface comprises a part-spherical convex surface and the medial tibial condylar surface comprises a part-spherical concave surface, the part-spherical surfaces being arranged to enable the medial femoral condyle to engage in sphere-in-sphere engagement with the medial tibial condyle, wherein the sphere-in-sphere engagement provides anterior-posterior stability of the femoral component relative to the tibial component;
wherein the lateral tibial condylar surface comprises a track surface for the lateral femoral condyle to move across as the medial condyle pivots around the sphere-in-sphere engagement; and
wherein the track surface is posteriorly unrestricted to permit the lateral femoral condylar surface to contact the track surface at a range of contact positions as the medial femoral condyle pivots relative to the medial tibial condyle around the sphere-in-sphere engagement.

2. A knee prosthesis according to claim 1 wherein the track surface extends from a position defined by the contact position between the lateral femoral condylar surface and the lateral tibial condylar surface at full extension to a position defined by the posterior extreme of the lateral tibial condylar surface.

3. A knee prosthesis according to claim 1, wherein the track surface comprises a planar portion which is planar along the track in the anterior-posterior direction.

4. A knee prosthesis according to claim 3, wherein the tibial component has a base which defines a plane and wherein the planar portion is parallel to the plane.

5. A knee prosthesis according to claim 3 wherein tibial component has a base which defines a plane and wherein the planar portion is inclined posteriorly downwards relative to the plane.

6. A knee prosthesis according to claim 5, wherein the planar portion is inclined up to 12 degrees posteriorly downwards relative to the plane of the base.

7. A knee prosthesis according to claim 3, wherein the planar portion extends in the anterior-posterior direction across the entire track surface.

8. A knee prosthesis according to claim 7, wherein the tibial component includes a lateral surface on the lateral side of the planar portion of the track surface, wherein the lateral surface is planar and on the same plane as the planar portion of the track surface.

9. A knee prosthesis according to claim 1, wherein the track surface comprises a posterior portion which is convex in sagittal section.

10. A knee prosthesis according to claim 1, wherein the medial-lateral profile of the track surface conforms with the medial-lateral profile of the lateral femoral condylar surface.

11. A knee prosthesis according to claim 1, wherein the track surface has a posterior extreme at a posterior extreme of the lateral tibial condylar surface, wherein the tibial component comprises a rounded portion which connects the posterior extreme of the track surface to the posterior surface of the lateral side of the tibial component, wherein the range of contact positions which the lateral femoral condylar surface can contact the track surface are selected from the group consisting of (i) a contact position at the posterior extreme of the track surface, (ii) a contact position on the rounded portion, and combinations thereof.

12. A knee prosthesis according to claim 1, wherein the tibial component has a base which defines a plane, wherein the posterior surface of the lateral side of the tibial component extends distally beyond the plane of the base.

13. A knee prosthesis according to claim 1, wherein the lateral femoral condylar surface is able to lose contact with the lateral tibial condylar surface.

14. A knee prosthesis according to claim 1, wherein the posterior surface of the lateral side of the tibial component is nearer to the medial-lateral axis than the posterior surface of the medial side of the tibial component.

15. A knee prosthesis according to claim 14, wherein the posterior surface of the lateral side of the tibial component is in a range from 5 mm to 9 mm nearer to the medial-lateral axis than the posterior surface of the medial side of the tibial component.

16. A knee prosthesis according to claim 14, wherein the posterior surface of the lateral side of the tibial component is about 7 mm nearer to the medial-lateral axis than the posterior surface of the medial side of the tibial component.

17. A knee prosthesis according to claim 1, wherein the track surface extends from a position defined by the contact position between the lateral femoral condylar surface and the lateral tibial condylar surface at full extension to a position defined by the posterior extreme of the lateral tibial condylar surface and wherein the anterior-posterior length of the track is in a range from 20 mm to 30 mm.

18. A knee prosthesis according to claim 1, wherein the tibial component has a base which defines a plane and wherein the medial tibial condylar surface has an anterior lip and a posterior lip which at least partly define the part-spherical concave surface, and wherein the anterior lip is higher than the posterior lip, when the height of each lip is measured from the plane of the base.

19. A knee prosthesis according to claim 18, wherein the anterior lip has a height of 7 mm to 13 mm and the posterior lip has a height in a range from 1 mm to 5 mm, when the height of each lip is measured from a plane which is parallel to the plane of the base and which contains the point on the concave surface nearest to the plane of the base.

20. A knee prosthesis according to claim 19, wherein the height of the anterior lip is in a range from 9 mm to 11 mm and the height of the posterior lip is in a range from 2 mm to 4 mm.

21. A knee prosthesis according to claim 19, wherein the height of the anterior lip is about 10 mm and the height of the posterior lip is about 3 mm.

22. A knee prosthesis according to claim 1, wherein the medial femoral condylar surface is circular in sagittal section with a radius in a range from 20 to 25 mm, wherein the tibial component has a base which defines a plane and wherein the point on the concave surface nearest to the plane of the base is a distance in a range from 20 to 25 mm from the posterior surface of the medial side of the tibial component.

23. A knee prosthesis according to claim 1, wherein a posterior part of the lateral femoral condylar surface is circular in sagittal section and has a radius which is smaller than the radius of the part-spherical convex surface of the femoral medial condyle.

24. A knee prosthesis according to claim 1, wherein the lateral tibial condylar surface has a concave anterior restriction arranged to contact a restriction part of the lateral femoral condylar surface in full extension.

25. A knee prosthesis according to claim 24, wherein the restriction part of the lateral tibial condylar surface is anterior to a posterior part of the lateral femoral condylar surface, wherein the posterior part is circular in sagittal section from its posterior end to a position which contacts the lateral tibial condylar surface in full extension, and wherein the restriction part of the lateral femoral condylar surface has a larger radius in sagittal section than the radius of the posterior part.

26. A knee prosthesis according to claim 1, wherein the lateral tibial condylar surface has planar anterior surface and the lateral femoral condylar surface has a planar formation which is arranged to contact the planar anterior surface of the lateral tibial condylar surface in full extension.

27. A knee prosthesis according to claim 26, wherein the planar formation on the lateral femoral condylar surface is anterior to a posterior part of the lateral femoral condylar surface which is circular in sagittal section from its posterior end to a position which contacts the lateral tibial condylar surface in full extension.

28. A knee prosthesis according to claim 26, wherein the lateral tibial condylar surface is planar from its anterior extreme to its posterior extreme.

29. A knee prosthesis according to any claim 1, wherein the tibial component comprises an intercondylar eminence between the lateral tibial condylar surface and the medial tibial condylar surface, wherein the lateral side of the intercondylar eminence joins the track surface and is profiled to allow the pivotable movement of the lateral femoral condyle across the track surface.

30. A knee prosthesis according to claim 29, wherein the lateral side of the intercondylar eminence comprises in plan view a part-circumferential profile.

31. A knee prosthesis according to claim 29, wherein the lateral side of the intercondylar eminence comprises in plan view a profile which is a combination of at least one tangential profile and at least one part-circumferential profile.

32. (canceled)

33. A method of implanting a knee prosthesis, comprising:
affixing a femoral component to a femur; and
affixing a tibial component to a tibia;
wherein the femoral component comprises a medial femoral condyle having a medial femoral condylar surface and a lateral femoral condyle having a lateral femoral condylar surface;
wherein the tibial component comprises a medial tibial condyle having a medial tibial condylar surface and a lateral tibial condyle having a lateral tibial condylar surface;
wherein the medial femoral condylar surface comprises a part-spherical convex surface and the medial tibial condylar surface comprises a part-spherical concave surface, the part-spherical surfaces being arranged to enable the medial femoral condyle to engage in sphere-in-sphere engagement with the medial tibial condyle, wherein the sphere-in-sphere engagement provides anterior-posterior stability of the femoral component relative to the tibial component;
wherein the lateral tibial condylar surface comprises a track surface for the lateral femoral condyle to move across as the medial condyle pivots around the sphere-in-sphere engagement; and
wherein the track surface is posteriorly unrestricted to permit the lateral femoral condylar surface to contact the track surface at a range of contact positions as the medial femoral condyle pivots relative to the medial tibial condyle around the sphere-in-sphere engagement.
